(19) United States
(12) Patent Application Publication
(10) Pub. No.: US 2023/0181687 A1
(43) Pub. Date: Jun. 15, 2023

(54) Methods and Compositions for Treatment of Age-Related Dysfunction

(71) Applicant: The University of Tokyo, Tokyo (JP)

(72) Inventors: Yuji Yamanashi, Tokyo (JP); Ryo Ueta, Tokyo (JP); Satoshi Sugita, Tochigi (JP); Yoshihiko Minegishi, Tochigi (JP)

(21) Appl. No.: 17/925,455

(22) PCT Filed: Jun. 3, 2021
(86) PCT No.: PCT/JP2021/021281

§ 371 Date: Nov. 15, 2022

Related U.S. Application Data
(60) Provisional application No. 63/034,506, filed on Jun. 4, 2020.

(51) Int. Cl. A61K 38/17; C12N 15/86; A61P 21/00

(52) U.S. Cl. CPC A61K 38/1719; C12N 15/86; A61P 21/00; C12N 2750/14143; C12N 2750/14145; C12N 2750/14151; A61K 48/00

(57) ABSTRACT

Disclosed herein are methods and compositions for the treatment of age-related dysfunction, particularly, age-related motor impairment.

### TECHNICAL FIELD

The field of the invention generally relates to methods and compositions for treatment of age-related dysfunction, particularly, age-related motor impairment in a subject in need thereof, particularly, an elderly subject without a disease associated with defective NMJs.

### BACKGROUND ART

Muscle denervation at the neuromuscular junction (NMJ), the essential synapse between motor neuron and skeletal muscle, is associated with age-related motor impairment. Therefore, improving muscle innervation at aged NMJs may be an effective therapeutic strategy for treating the impairment. We previously demonstrated that the muscle protein Dok-7 plays an essential role in NMJ formation, and, indeed, its forced expression in muscle enlarges NMJs. Moreover, therapeutic administration of an adeno-associated virus vector encoding human Dok-7 (DOK7 gene therapy) suppressed muscle denervation and enhanced motor activity in a mouse model of amyotrophic lateral sclerosis (ALS).

### SUMMARY OF INVENTION

Surprisingly, the present inventors found that DOK7 gene therapy significantly enhances motor function and muscle strength together with NMJ innervation in aged mice. Furthermore, the treated mice showed greatly increased compound muscle action potential (CMAP) amplitudes compared to the controls, suggesting enhanced neuromuscular transmission. Thus, therapies aimed at enhancing NMJ innervation have potential for treating age-related dysfunction, particularly, age-related motor impairment.

Age-related decline in motor function has a major impact on quality of human life (Hunter et al, 2016). The motor impairment involves age-related changes at least in the nerve and muscle systems, including a pathogenic loss of skeletal muscle mass and strength, known as sarcopenia.

Accumulating evidence raises the possibility that the age-related decline in motor function is caused, at least in part, by functional impairment of the neuromuscular junction (NMJ), a cholinergic synapse essential for motoneural control of skeletal muscle contraction (Gonzalez-Freire et al, 2014; Liu et al, 2017; Punga and Ruegg, 2012, Tintignac et al, 2015).

Studies with rodents demonstrated age-related denervation at NMJs in addition to degeneration of the presynaptic motor nerve terminals, where the neurotransmitter acetylcholine is released, and the postsynaptic endplate, where acetylcholine receptors (AChRs) densely cluster, suggesting an impaired neuromuscular transmission with ageing (Chai et al, 2011; Valdez et al, 2010). In humans, electrophysiological and muscle fiber-type studies suggested age-related denervation at NMJs (Campbell et al, 1973; Lexell and Downham, 1991; Spendiff et al, 2016). Indeed, it is reported that the denervation rate at NMJs increases upon ageing, although age-related morphological changes at NMJs remain controversial (see below; Jones et al, 2017; Oda, 1984; Wokke et al, 1990). Moreover, a recent study suggests that the increased rate of NMJ denervation contributes to the reduction in muscle strength in patients with sarcopenia (Piasecki et al, 2018), supporting the idea that the NMJ is a possible therapeutic target for treating age-related dysfunction, particularly, age-related motor dysfunction.

In mammals, the muscle-specific receptor tyrosine kinase MuSK is essential for the formation and maintenance of NMJs (Burden, 2002). The receptor kinase is activated by the motor neuron-derived agrin, which binds to MuSK's coreceptor, low-density lipoprotein receptor-related protein 4 (Lrp4) (Kim et al, 2008; Zhang et al, 2008). Furthermore, activation of MuSK also requires Dok-7 (downstream of tyrosine kinases-7) (Inoue et al, 2009; Okada et al, 2006).

Indeed, biallelic mutations in the human DOK7 gene cause a limb-girdle type of congenital myasthenic syndrome (DOK7 myasthenia), a disorder characterized by defective NMJ structure or NMJ synaptopathy (Beeson et al, 2006). In addition, we previously generated AAV-D7, a recombinant muscle-tropic adeno-associated virus (AAV) serotype 9 vector carrying the human DOK7 gene under the control of the cytomegalovirus promoter, and demonstrated that therapeutic administration of AAV-D7—DOK7 gene therapy—enlarges NMJs, improves the impaired motor activity, and ameliorates the shortened lifespan in mouse models of DOK7 myasthenia and autosomal dominant Emery-Dreifuss muscular dystrophy, a disease associated with defective NMJs due to genetic mutations in the lamin A/C gene (Arimura et al, 2014; Méjat et al, 2009). Moreover, DOK7 gene therapy suppressed denervation at NMJs, and enhanced motor activity and life span in a mouse model of familial amyotrophic lateral sclerosis (ALS), a fatal neuromuscular disease with motor neuron degeneration (Miyoshi et al, 2017). These findings demonstrate potential for DOK7 gene therapy in various motor neuron diseases as well as myopathies with NMJ defects.

Given that NMJ denervation appears to play a crucial role in age-related decline in motor function similar to that observed in ALS model mice (Valdez et al, 2012), DOK7 gene therapy might also ameliorate age-related motor impairment, by suppressing denervation at NMJs. Thus, in the present study, we examined whether DOK7 gene therapy improves the motor function in aged mice.

In some embodiments, the present invention is directed to a method for the treatment of age-related dysfunction, particularly, age-related motor impairment in a subject in need thereof, the method comprising administering to the subject a therapeutically effective amount of an NMJ (neuromuscular junction) targeting agent.

### BRIEF DESCRIPTION OF DRAWINGS

This invention is further understood by reference to the drawings wherein:

FIG. 1. AAV-D7 treatment activates MuSK and enhances NMJ innervation in aged mice. Twenty-four month-old (mo) male mice were treated with AAV-D7 or the control empty vector (AAV-ø), and subjected to the following assays at 28 months of age (4 months after the administration of AAV). (A) Anti-MuSK or anti-AChR betal immunoprecipitates (IP: MuSK or IP: AChR betal respectively) from the hindlimb-muscle lysates of non-treated (−) 24 mo mice, or 28 mo mice treated with AAV-D7 or AAV-ø, together with whole-tissue lysates (WTLs), were subjected to immunoblotting (IB) with the indicated antibodies. P-Tyr, phosphotyrosine. (B-E) Longitudinal sections of tibialis anterior (TA) muscles of non-treated (NT) 24 mo mice or 28 mo mice treated with AAV-D7 or AAV-ø were stained with alpha-bungarotoxin (AChR) and antibodies to synapsin-1 (Nerve terminal), and representative images are shown (B) (Scale bar, 50 μm). The area of AChR clusters (C) and motor nerve terminals (D), and the percentage of denervated NMJs (E) were quantified. Error bars indicate means±SEM (n=15 mice for NT 24 mo mice; n=8 mice for AAV-ø-treated 28 mo mice; n=8 mice for AAV-D7-treated 28 mo mice). *P<0.05, **P<0.01, ***P<0.001 by one-way ANOVA followed by Student's t-test post-hoc analysis. N.S., not significant.

FIG. 2. AAV-D7 treatment enhances the maximal amplitudes of CMAPs in aged mice. Twenty-four mo male mice were treated with AAV-D7 or the control empty vector (AAV-ø), and the maximal amplitudes of compound muscle action potentials (CMAPs) of TA muscles at 10 Hz stimulations were measured at 24 months of age (NT, non-treated) or 28 months of age (AAV-D7- or AAV-ø-treated). Representative traces (A) and quantitative data (B) are shown. Error bars indicate means±SEM (n=15 mice for NT 24 mo mice; n=10 mice for AAV-ø-treated 28 mo mice; n=14 mice for AAV-D7-treated 28 mo mice). *P<0.05 by one-way ANOVA followed by Student's t-test post-hoc analysis.

FIG. 3. AAV-D7 treatment enhances motor function and muscle strength in aged mice. Twenty-four mo male mice were treated with AAV-D7 or AAV-ø, and subjected to the following assays at the indicated ages. (A and B) Motor performance of pre-dose aged mice or those treated with AAV-D7 or AAV-ø were evaluated by rotarod test at the indicated ages (pre-dose mice, 24 months of age; treated mice, 24.5 to 26.5 months of age). Each score is expressed as relative value (%) of the latency to fall off the rotarod to pre-dose value of the individual mice at 24 months of age (A), and the absolute values of pre-dose mice are shown (B). Error bars indicate means±SEM (n=20 mice for AAV-ø-treated mice; n=21 mice for AAV-D7-treated mice). (C) Body weight of pre-dose aged mice or those treated with AAV-D7 or AAV-ø at the indicated ages (pre-dose mice, 24 months of age; treated mice, 24.5 to 26.5 months of age) was measured. Error bars indicate means±SEM (n=20 mice for AAV-ø-treated mice; n=21 mice for AAV-D7-treated mice). There is no statistically significant difference between the two tested groups at each time point (Student's t-test). (D and E) Maximal isometric torque of pre-dose aged mice at 24 months of age or those treated with AAV-D7 or AAV-ø at 28 months of age (4 months after the administration of AAV) was measured. Each score is expressed as relative value (%) of the plantarflexion torque to pre-dose value of the individual mice at 24 months of age (D), and the absolute values of pre-dose mice are shown (E). Error bars indicate mean±SEM (n=7 mice for AAV-ø-treated mice; n=9 mice for AAV-D7-treated mice). (F and G) Transverse sections of TA muscle of 28 mo mice treated with AAV-D7 or AAV-ø were stained with hematoxylin and eosin (F), and the myofiber cross-sectional areas (CSA) were quantified (G). Scale bar, 100 μm. Error bars indicate means±SEM (n=8 mice for AAV-ø-treated mice; n=8 mice for AAV-D7-treated mice). *P<0.05, **P<0.01, ***P<0.001, N.S., not significant by Student's t-test. #P<0.05, ##P<0.01, ###P<0.001 by paired Student's t-test.

FIG. 4 illustrates an exemplary mechanism for the treatment of age-related dysfunction with AAV-D7.

### DESCRIPTION OF EMBODIMENTS

DNA

The term “DNA” herein may be either a sense strand or an antisense strand (for example, one which can be used as a probe), and the form may be either single stranded or double stranded. Also, it may be either a genomic DNA or a cDNA, or alternatively may be a synthesized DNA.

The DNA according to the most preferred embodiment of the present invention is a DNA having the nucleotide sequence set out in SEQ ID NO: 2 which encodes Dok-7, and the DNA of the present invention may further include a variety of mutants and homologues that modulate the motor activity.

The mutants and homologues of the DNA having the nucleotide sequence set out in SEQ ID NO: 2 include, for example, DNAs having a nucleotide sequence which can hybridize with the nucleotide sequence set out in SEQ ID NO: 2 under stringent conditions. The “stringent conditions” herein may include, for example, conditions of allowing a reaction in a common hybridization buffer at 40 to 70° C. (preferably, 60 to 65° C.), and washing in a washing fluid having a salt concentration of 15 to 300 mM (preferably, 15 to 60 mM).

In addition, DNAs including a nucleotide sequence encoding an amino acid sequence having the substitution, deletion and/or addition of one or several amino acid residues in the amino acid sequence set out in SEQ ID NO: 1 (DOK7) are also included in the present invention. Herein, the term “one or several” means usually within 50 amino acids, preferably within 30 amino acids, more preferably within 10 amino acids (for example, within 5 amino acids, within 3 amino acids, and 1 amino acid). For maintaining the ability to activate a muscle-specific tyrosine kinase, the mutation of the amino acid residue is preferably conducted with another amino acid having a conserved property of the amino acid side chain. For example, in terms of the property of the amino acid side chains, the amino acids include hydrophobic amino acids (A, I, L, M, F, P, W, Y, V), hydrophilic amino acids (R, D, N, C, E, Q, G, H, K, S, T), amino acids having an aliphatic side chain (G, A, V, L, I, P), amino acids having a hydroxyl group-containing side chain (S, T, Y), amino acids having a sulfur atom-containing side chain (C, M), amino acids having a carboxylic acid and an amide-containing side chain (D, N, E, Q), amino acids having a base-containing side chain (R, K, H), and amino acids having an aromatic group-containing side chain (H, F, Y, W). In the parentheses, each represents one-letter code for the amino acid.

It has been already known that proteins having an amino acid sequence modified by deletion or addition of one or several amino acid residues, and/or substitution with other amino acid in a certain amino acid sequence maintain their original biological activity (Mark, D. F. et al., Proc. Natl. Acad. Sci. USA (1984) 81, 5662-5666; Zoller, M. J. & Smith, M. Nucleic Acids Research (1982) 10, 6487-6500; Wang, A. et al., Science 224, 1431-1433; Dalbadie-McFarland, G. et al., Proc. Natl. Acad. Sci. USA (1982) 79, 6409-6413).

According to one preferred embodiment of the DNA of the present invention, the DNA encodes a polypeptide that has the substitution, deletion and/or addition of one or several amino acid residues in the amino acid sequence of positions 1 to 230 set out in SEQ ID NO: 1 (but has the amino acid sequence of positions 1 to 60 set out in SEQ ID NO: 1), and that modulates the motor activity.

Furthermore, the mutants and homologues of the DNA having the nucleotide sequence set out in SEQ ID NO: 2 include DNAs including a nucleotide sequence that has high homology to the nucleotide sequence set out in SEQ ID NO: 2. Such DNAs have homology of preferably 90% or higher, and more preferably 95% or higher (96% or higher, 97% or higher, 98% or higher, or 99% or higher) to the nucleotide sequence set out in SEQ ID NO: 2 of Sequence Listing. The homology of the amino acid sequence and the nucleotide sequence can be determined with the BLAST algorithm (Proc. Natl. Acad. Sci. USA 90: 5873-5877, 1993) by Karlin and Altschul. On the basis of this algorithm, programs named BLASTN, BLASTX etc. have been developed (Altschul et al. J. Mol. Biol. 215: 403-410, 1990). When the nucleotide sequence is analyzed with BLASTN on the basis of BLAST, the parameter may include, for example, score=100, and wordlength=12. Alternatively, when the amino acid sequence is analyzed with BLASTX on the basis of BLAST, the parameter may include, for example, score=50, and wordlength=3. When BLAST and Gapped BLAST program are employed, default parameters of each program may be used. Specific processes in these analysis method have been known (HyperTextTransferProtocol://WorldWideWeb.ncbi.nlm.nihDOTgov, wherein HyperTextTransferProtocol=“http”, WorldWideWeb=“www”, and DOT=“.”).

The method for obtaining the DNA of the present invention is not particularly limited, and includes known methods such as methods for obtaining a cDNA by reverse transcription from a mRNA (for example, RT-PCR method), methods of preparing from a genomic DNA, methods of synthesizing by chemical synthesis, methods of isolating from a genomic DNA library or a cDNA library, and the like (see, for example, Japanese Unexamined Patent Application, First Publication No. Hei 11-29599).

Vector

The vector of the present invention can be produced by inserting the DNA described above into an adequate vector.

The “adequate vector” may be one capable of self-proliferating or keeping replicating in a variety of host of a prokaryotic organism and/or eukaryotic organism, and can be selected appropriately depending on the intended use thereof. For example, when obtaining a large amount of the DNA is desired, a high-copy vector can be selected, whereas an expression vector can be selected when obtaining a polypeptide is intended.

Transformant

The transformant of the present invention can be produced by introducing the vector including the DNA described above into a host.

Such a host is not particularly limited as long as it is compatible with and can be transformed with the vector of the present invention, and specific examples thereof include known naturally occurring cells such as bacteria, yeast, animal cells and insect cells, as well as artificially established cells.

The method for introducing the vector may be selected appropriately depending on the type of the vector, the host, and the like. Specific examples of the method include known methods such as a protoplast method, a competent method, and the like, but not particularly limited thereto.

Polypeptide

The polypeptide of the present invention can be produced using, for example, a transformant into which an expression vector including the DNA described above was introduced. In other words, the transformant is first cultured under appropriate conditions, whereby the protein (polypeptide) encoded by this DNA is synthesized. Accordingly, the polypeptide of the present invention can be obtained by recovering the synthesized protein from the transformant or culture fluid.

The transformant can be cultured through appropriately selecting a known nutrient medium depending on the type of the transformant and the like such that the polypeptide can be readily obtained in a large amount, and then appropriately adjusting the temperature, pH, culture time and the like of the nutrient medium.

The method of isolation and the method of purification of the polypeptide are not particularly limited, and examples thereof include known methods such as a method in which solubility is utilized, a method in which the difference in the molecular weight is utilized, a method in which the charging is utilized, and the like.

Antibody, Antibody Fragment

The antibody or the antibody fragment of the present invention binds to the polypeptide of the present invention described above.

The antibody of the present invention may be either a polyclonal antibody, or a monoclonal antibody. Moreover, the antibody includes antisera obtained by immunizing an animal for immunization such as a rabbit with the polypeptide of the present invention, polyclonal antibodies and monoclonal antibodies in all classes, human antibodies and humanized antibodies prepared by gene recombination, and variously modified antibodies.

The method for producing the antibody of the present invention includes, for example, a conventionally known hybridoma technique (Kohler and Milstein, Nature 256: 495 (1975)),In addition, the antibody fragment of the present invention includes Fab, F (ab′)2, Fv, or single chain Fv (scfv) in which Fv of an H chain and an L chain are linked with a suitable linker (Huston, J. S. et al., Proc. Natl. Acad. Sci. U.S.A. (1988) 85, 5879-5883).

Therapeutic Treatments

In Vitro Expansion for Transplantation

The methods described herein may be used to expand cells, such as stem cells and HSPCs, in vitro for allogenic or autologous transplantation in subjects. Subjects who may be in need of such treatment include those suffering from hematological diseases, cancers, immunodeficiencies, and the like. For example, stem cells obtained from umbilical cord blood (UCB) and expanded in vitro in an undifferentiated state and then the expanded cells may be transplanted in a subject in need thereof. In some embodiments, the methods described herein are used to expand engineered T cells, which may express chimeric antigen receptors against given antigens such as tumor-associated antigens and HIV antigens, and then the expanded cells are transplanted in a subject.

In some embodiments, the cells to be transplanted in a subject are contacted with exogenous Egfl7 by, e.g., culturing the cells in culture medium that is supplemented with Egfl7 or by overexpressing Egfl7 in feeder layer cells (e.g., endothelial cells like (human umbilical vein endothelial cells (HUVECs) or fibroblasts) using an Egfl7 expression vector. In some embodiments, expression of the cells to be transplanted in a subject are contacted with a mutant RGDdel protein.

Treatment of Diseases

As set forth in the detailed examples, HSPCs such as ETPs and ECs in subjects suffering from irradiation-induced myelosuppression can be expanded in vivo using the methods disclosed herein. Therefore, the methods disclosed herein may be used to treat subjects suffering from irradiation-induced myelosuppression, HSC associated diseases such as sickle cell anemia, T cell deficiencies resulting from HIV infection, chemotherapy, organ-transplantation, bone marrow transplantation, and aging. In some embodiments, the treatment methods comprise increasing Egfl7 expression in the HSPCs in the subject and/or administering to the subject a therapeutically effective amount of one or more Egfl7 proteins. In some embodiments, the treatment methods comprise administering to the subject a therapeutically effective amount of one or more mutant RGDdel proteins. In some embodiments, the treatment methods further comprise administering to the subject a therapeutically effective amount of an Itgb3 inhibitor.

In some embodiments, cells, e.g., stem cells, in a subject are induced to proliferate in vivo by administering to the subject an exogenous amount of Egfl7 or a mutated Egfl7 protein that lacks the RGD domain of the corresponding wildtype Egfl7 protein. In some embodiments, cells, e.g., stem cells, in a subject are induced to proliferate in vivo by administering to the subject an expression vector that expresses Egfl7 or a mutant RGDdel protein. In some embodiments, the cells in a subject that are induced to proliferate are endogenous cells. In some embodiments, the cells in a subject that are induced to proliferate are exogenous cells that have been transplanted in the subject. In some embodiments, the exogenous cells are autologous. In some embodiments, the exogenous cells are allogenic. In some embodiments, the exogenous cells are syngeneic.

Kits

In some embodiments, the present invention provides kits comprising one or more Notch activators (e.g., Egfl7 proteins such as human Egfl7 or a mutant RGDdel protein).

In some embodiments, the present invention provides kits comprising one or more NMJ targeting agents, such as a polynucleotide which encodes Dok-7, optionally in a composition or in combination with one or more supplementary agents, packaged together with one or more reagents or drug delivery devices for administering the one or more NMJ targeting agents to a subject. In some embodiments, the kits comprise the one or more NMJ targeting agents, optionally in one or more unit dosage forms, packaged together as a pack and/or in drug delivery device, e.g., a pre-filled syringe.

In some embodiments, the kits include a carrier, package, or container that may be compartmentalized to receive one or more containers, such as vials, tubes, and the like. In some embodiments, the kits optionally include an identifying description or label or instructions relating to its use. In some embodiments, the kits include information prescribed by a governmental agency that regulates the manufacture, use, or sale of compounds and compositions according to the present invention.

Compositions

Compositions of the present invention, including pharmaceutical compositions, include one or more NMJ targeting agents. As used herein, an “NMJ targeting agent” refers to a compound or composition that increases the formation of NMJs, e.g., a polynucleotide which encodes Dok-7, or analogs or homologs thereof. In some embodiments, the compositions may further comprise a non-NMJ-targeting therapeutic(s), e.g., which induce muscle hypertrophy.

The term “pharmaceutical composition” refers to a composition suitable for pharmaceutical use in a subject. A pharmaceutical composition generally comprises an effective amount or a therapeutically effective amount of an active agent, e.g., one or more NMJ targeting agents, and a pharmaceutically acceptable carrier. Pharmaceutical compositions according to the present invention may further include one or more supplementary agents.

One or more NMJ targeting agents may be administered, preferably in the form of pharmaceutical compositions, to a subject. Preferably the subject is mammalian, more preferably, the subject is human, preferably, an elderly subject without a disease associated with defective NMJs. Preferred pharmaceutical compositions are those comprising at least one NMJ targeting agent in a therapeutically effective amount and a pharmaceutically acceptable vehicle.

As used herein, an “elderly” subject refers to a subject aged 60 years, 65 years, 70 years, or 75 years, or older.

As used herein, an “effective amount” refers to a dosage or amount sufficient to produce a desired result. The desired result may comprise an objective or subjective improvement in the recipient of the dosage or amount, e.g., long-term survival, effective prevention of a disease state, and the like.

As used herein, a “therapeutically effective amount” refers to an amount that may be used to treat, prevent, or inhibit a given disease or condition, in a subject as compared to a control, such as a placebo. The skilled artisan will appreciate that certain factors may influence the amount required to effectively treat a subject, including the degree of the disease or condition, previous treatments, the general health and age of the subject, and the like. Nevertheless, therapeutically effective amounts may be readily determined by methods in the art. It should be noted that treatment of a subject with a therapeutically effective amount may be administered as a single dose or as a series of several doses. The dosages used for treatment may increase or decrease over the course of a given treatment. Optimal dosages for a given set of conditions may be ascertained by those skilled in the art using dosage-determination tests and/or diagnostic assays in the art. Dosage-determination tests and/or diagnostic assays may be used to monitor and adjust dosages during the course of treatment.

Pharmaceutical compositions of the present invention may be formulated for the intended route of delivery, including intravenous, intramuscular, intra peritoneal, subcutaneous, intraocular, intrathecal, intraarticular, intrasynovial, cisternal, intrahepatic, intralesional injection, intracranial injection, infusion, and/or inhaled routes of administration using methods known in the art. Pharmaceutical compositions according to the present invention may include one or more of the following: pH buffered solutions, adjuvants (e.g., preservatives, wetting agents, emulsifying agents, and dispersing agents), liposomal formulations, nanoparticles, dispersions, suspensions, or emulsions, as well as sterile powders for reconstitution into sterile injectable solutions or dispersions. The compositions and formulations may be optimized for increased stability and efficacy using methods in the art.

The compositions of the present invention may be administered to a subject by any suitable route including oral, transdermal, subcutaneous, intranasal, inhalation, intramuscular, and intravascular administration. It will be appreciated that the preferred route of administration and pharmaceutical formulation will vary with the condition and age of the subject, the nature of the condition to be treated, the therapeutic effect desired, and the particular NMJ targeting agent used.

As used herein, a “pharmaceutically acceptable vehicle” or “pharmaceutically acceptable carrier” are used interchangeably and refer to solvents, dispersion media, coatings, antibacterial and antifungal agents, isotonic and absorption delaying agents, and the like, that are compatible with pharmaceutical administration and comply with the applicable standards and regulations, e.g., the pharmacopeial standards set forth in the United States Pharmacopeia and the National Formulary (USP-NF) book, for pharmaceutical administration. Thus, for example, unsterile water is excluded as a pharmaceutically acceptable carrier for, at least, intravenous administration. Pharmaceutically acceptable vehicles include those known in the art. See, e.g., Remington: The Science and Practice of Pharmacy. 20^{th} ed. (2000) Lippincott Williams & Wilkins. Baltimore, Md., which is herein incorporated by reference.

The pharmaceutical compositions of the present invention may be provided in dosage unit forms. As used herein, a “dosage unit form” refers to physically discrete units suited as unitary dosages for the subject to be treated; each unit containing a predetermined quantity of the one or more NMJ targeting agents calculated to produce the desired therapeutic effect in association with the required pharmaceutically acceptable carrier. The specification for the dosage unit forms of the invention are dictated by and directly dependent on the unique characteristics of the given NMJ targeting agent and desired therapeutic effect to be achieved, and the limitations inherent in the art of compounding such an active compound for the treatment of individuals.

Toxicity and therapeutic efficacy of NMJ targeting agents according to the instant invention and compositions thereof can be determined using cell cultures and/or experimental animals and pharmaceutical procedures in the art. For example, one may determine the lethal dose, LC₅₀ (the dose expressed as concentration×exposure time that is lethal to 50% of the population) or the LD₅₀ (the dose lethal to 50% of the population), and the ED₅₀ (the dose therapeutically effective in 50% of the population) by methods in the art. The dose ratio between toxic and therapeutic effects is the therapeutic index and it can be expressed as the ratio LD₅₀/ED₅₀. NMJ targeting agents which exhibit large therapeutic indices are preferred. While NMJ targeting agents that result in toxic side-effects may be used, care should be taken to design a delivery system that targets such compounds to the site of treatment to minimize potential damage to uninfected cells and, thereby, reduce side-effects.

The data obtained from the cell culture assays and animal studies can be used in formulating a range of dosages for use in humans. Preferred dosages provide a range of circulating concentrations that include the ED₅₀ with little or no toxicity. The dosage may vary depending upon the dosage form employed and the route of administration utilized. Therapeutically effective amounts and dosages of one or more NMJ targeting agents can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. Additionally, a dosage suitable for a given subject can be determined by an attending physician or qualified medical practitioner, based on various clinical factors.

The following examples are intended to illustrate but not to limit the invention.

### EXAMPLES

### Example 1

### DOK7 Gene Therapy Enhances NMJ Innervation in Aged Mice

Several mouse models with an accelerated ageing phenotype have been developed, enabling studies of motor dysfunction and muscle weakness as ageing-like phenotypes (Bütikofer et a1,2011; Didier et al, 2012; Hirofuji et al, 2000). However, it remains unclear whether the morphological and functional alterations of NMJs in these mouse models faithfully represent those in aged mice. C57BL/6 mice exhibit morphological NMJ abnormalities and decreased motor function at 24 months of age as compared to those in adulthood (e.g. at 2 or 6 months of age) (Cheng et al, 2013; Graber et al, 2013; Valdez et al, 2010). Because approximately 25% and 90% of C57BL/6 mice have been reported to die by 24 months and 32 months of age, respectively (Turturro, 1999), we defined mice at 24 months of age or older as aged mice in the current study. Indeed, other groups also utilized 24 month-old (“mo” hereafter) mice as aged mice for their studies on age-related alterations of NMJs and motor function (Andonian, 1987; Valdez et al, 2010). In addition, it has been reported that motor function declines over time even after 24 months of age (Cheng et al, 2013; Graber et al, 2013).

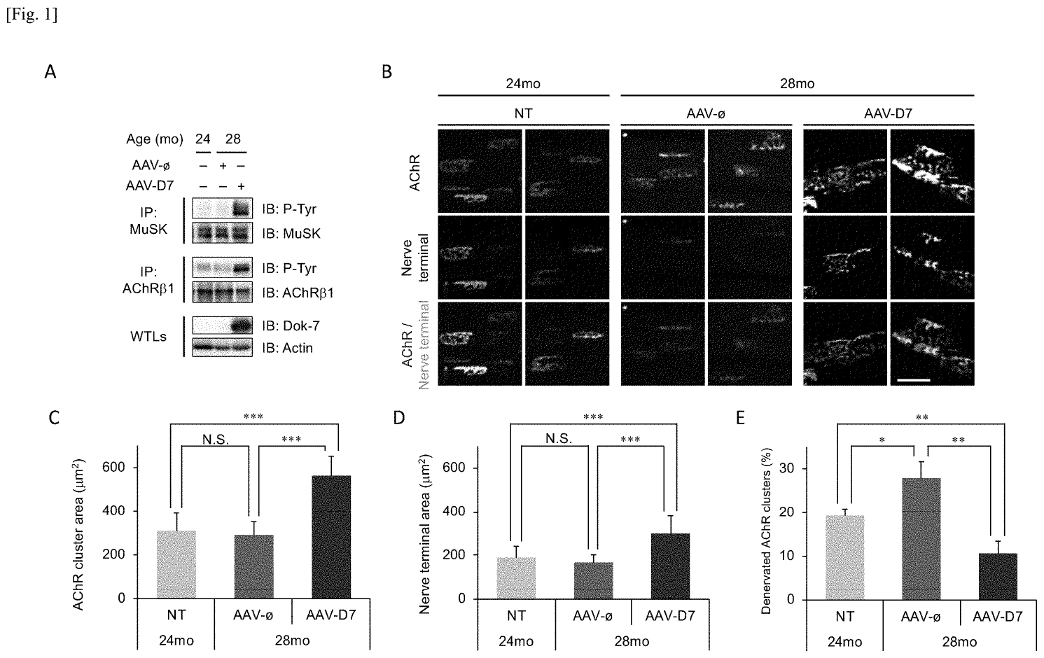

Here, we first examined whether therapeutic administration of AAV-D7 (DOK7 gene therapy) enhances the activation of MuSK in muscle and the subsequent formation of NMJs in aged mice. AAV-D7 or control empty vector (AAV-ø) was intravenously administered to 24 mo male mice with a single dose of 4.8×10¹³ viral genomes per kilogram of body weight (vg/kg BW). Four months after the administration, we confirmed that, compared with non-treated 24 mo and AAV-ø-treated 28 mo mice, AAV-D7-treated 28 mo mice showed robust enhancement of MuSK activation, as judged by phosphorylation of MuSK and AChR in the hindlimb muscle (FIG. 1A). Phosphorylation of the latter is known to be triggered by activation of MuSK (Fuhrer et al, 1997). To evaluate the effect of AAV-D7 treatment on the morphology of NMJs in aged mice, we stained frozen sections of the tibialis anterior (TA) muscle with alpha-bungarotoxin (alpha-Btx) to visualize AChR clusters (endplates) in myofibers, and with antibodies against synapsin-1 to label presynaptic motor nerve terminals (FIG. 1B). Consistent with our previous reports on much younger mice (Arimura et al, 2014; Miyoshi et al, 2017), AAV-D7 treatment significantly increased the postsynaptic area characterized by clustered AChRs as well as the area of presynaptic, synapsin-1-positive motor nerve terminals at NMJs (FIGS. 1C and 1D), demonstrating that AAV-D7 treatment enlarges NMJs in aged mice. It is of note that a gradual increase in the percentage of denervated NMJs is one of the typical alterations of NMJs with ageing in mice, as it is in a mouse model of ALS (Valdez et al, 2010; Valdez et al, 2012). Since AAV-D7 treatment not only enlarges NMJs but also suppresses NMJ denervation in ALS model mice (Miyoshi et al, 2017), we asked whether AAV-D7 treatment might also suppress NMJ denervation in aged mice. Indeed, while the percentage of denervated NMJs in AAV-ø-treated 28 mo mice increased 4 months after the administration of AAV, AAV-D7-treated 28 mo mice showed greatly reduced NMJ denervation compared with AAV-ø-treated 28 mo mice (FIG. 1E). Moreover, AAV-D7-treated mice showed significantly lower denervation than non-treated 24 mo mice. Together, these findings indicate that DOK7 gene therapy activates MuSK, enlarges NMJs, and enhances NMJ innervation in aged mice.

### Example 2

### DOK7 Gene Therapy Enhances Compound Muscle Action Potential Amplitudes in Aged Mice

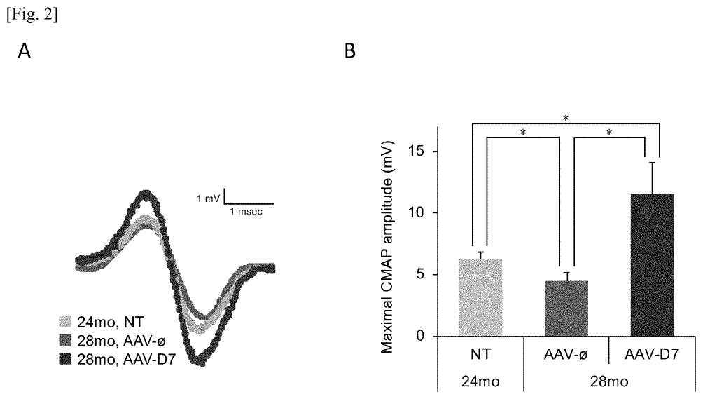

Motor neurons, when electrically excited by a nerve impulse, release the neurotransmitter acetylcholine to activate AChRs clustered on the postsynaptic membrane at the NMJ, leading to the generation of compound muscle action potentials (CMAPs), the amplitude of which depends on the number of myofibers firing action potentials (Willadt et al, 2018). Of note, several studies have reported that the maximal amplitude of CMAPs decreases with ageing in mammals (Kurokawa et al, 1999; Pannérec et al, 2016), consistent with the increased percentage of denervated NMJs (Wu et al 1996). Since NMJ innervation in aged mice was enhanced by AAV-D7 treatment (FIG. 1E), we hypothesized that neuromuscular transmission and subsequent firing of muscle action potentials would also be enhanced in AAV-D7-treated 28 mo mice. Thus, we tested whether the maximal amplitude of CMAPs increases upon the AAV-D7 treatment in aged mice. The CMAPs were obtained with recording electrodes inserted into the TA muscle upon sciatic nerve stimulation. While the maximal amplitude of CMAPs in control AAV-ø-treated 28 mo mice was lower than that in non-treated 24 mo mice, AAV-D7-treated 28 mo mice displayed a higher amplitude compared with non-treated 24 mo and AAV-ø-treated 28 mo mice (FIG. 2). Together with the enhanced NMJ innervation in AAV-D7-treated mice (FIG. 1E), these results indicate that AAV-D7 treatment enhances muscle action potentials evoked by nerve stimulation in aged mice, most likely due to enhanced neuromuscular transmission.

### Example 3

### DOK7 Gene Therapy Enhances Motor Function and Muscle Strength in Aged Mice

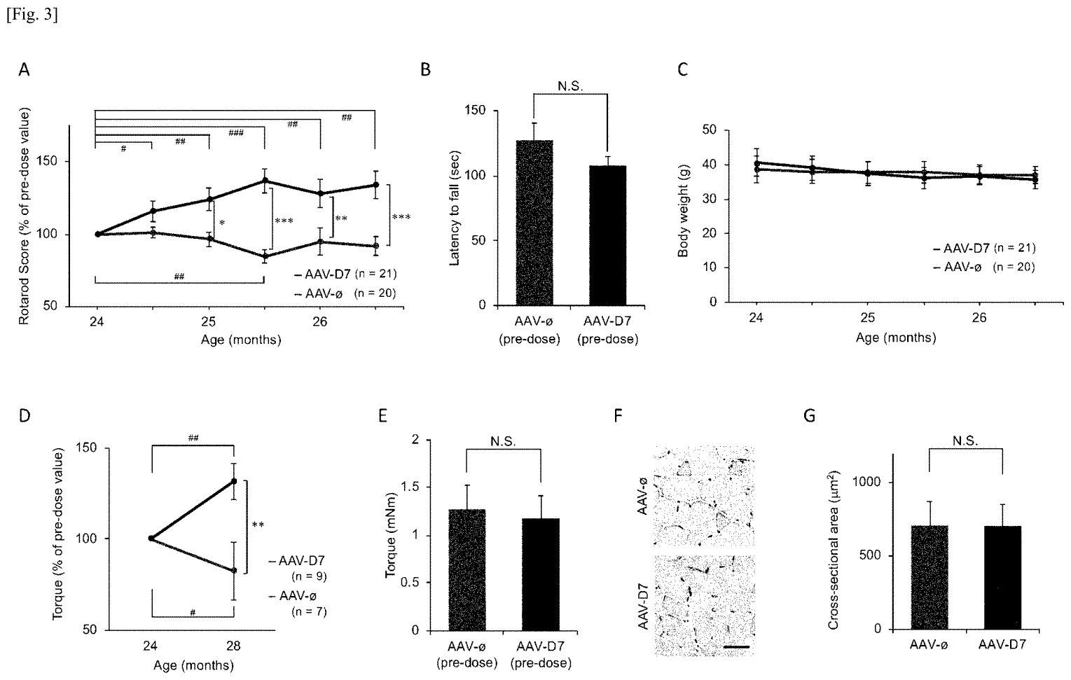

To determine whether AAV-D7 treatment improves motor function in aged mice, we compared the motor performance of AAV-D7-treated mice with that of AAV-ø-treated mice using the rotarod test, in which the latency of mice to fall off the rod rotating under continuous acceleration was measured. FIG. 3A shows that AAV-D7-treated mice significantly outperformed AAV-ø-treated mice in motor performance at each time point from 25 to 26.5 months of age (from 1 to 2.5 months after the administration of AAV), while no significant difference was observed between the two tested groups before AAV-ø or AAV-D7 treatment at 24 months of age (FIG. 3B). Furthermore, although the rotarod test score in AAV-ø-treated mice was comparable to or lower than that before treatment (at 24 months of age) at each time point throughout the test period, AAV-D7-treated mice showed a significant increase in their motor performance even at 24.5 months of age, or 0.5 months after treatment, in comparison to that before treatment. Given that no significant difference was observed in body weight between the AAV-ø- and AAV-D7-treatment groups throughout the test period (FIG. 3C), these results together indicate that DOK7 gene therapy enhances motor function in aged mice.

However, given that the motor performance determined by rotarod test (the ability of mice to maintain their balance on the rotating rod) is affected by many factors including altered cerebellar or spinal coafunction (Gunther et al, 2012; Sadakata et al, 2007), we cannot completely exclude the possibility that DOK7 gene therapy improved motor performance in aged mice via altered function of non-skeletal muscle tissues. Thus, to directly test muscle strength, we measured the in vivo twitch force of hindlimb muscle upon electrical stimulation. By directly stimulating gastrocnemius muscle using electrodes in contact with skin over the muscle, we measured the force of plantarflexor muscles with minimal invasiveness before and after administration of AAV-D7 or AAV-ø. As evident in FIG. 3D, AAV-D7-treated 28 mo mice showed significantly higher muscle strength than AAV-ø-treated 28 mo mice, while no significant difference was observed between pre-dose twitch forces of the two tested groups obtained before AAV-ø or AAV-D7 treatment at 24 months of age (FIG. 3E). Furthermore, the muscle strength in AAV-ø- or AAV-D7-treated 28 mo mice was reduced to 83% or increased to 132%, significantly and respectively, as compared to each strength before treatment (at 24 months of age) (FIG. 3D). Together these results indicate that DOK7 gene therapy enhances both motor function and muscle strength in aged mice.

Consistent with numerous studies demonstrating that denervation induces not only loss of NMJs, but also muscle atrophy (Guth et al, 1964), AAV-D7 treatment suppressed NMJ denervation and myofiber atrophy in a mouse model of ALS (Miyoshi et al, 2017). Thus, we thought that DOK7 gene therapy would suppress the loss of muscle mass in aged mice, and examined transverse sections of TA muscle in AAV-ø- or AAV-D7-treated 28 mo mice (FIG. 3F). However, the myofiber cross-sectional areas (CSA) were comparable between them (FIG. 3G), indicating that the enhancement of motor function and muscle strength does not need detectable muscle hypertrophy. Given that muscle strength is affected not only by muscle mass but also by its mechanical property (Gonzalez et al, 2000), AAV-D7 treatment may enhance muscle strength by altering an as yet unidentified mechanical property of myofibers in aged mice (see below).

A growing body of evidence indicates that structural and functional alterations of NMJs upon ageing contribute to age-related muscle weakness, highlighting the NMJ as a potential therapeutic target for age-related motor dysfunction (Taetzsch and Valdez, 2018; Willadt et al, 2018). However, by contrast with intensive studies performed on the effects of ageing on rodent NMJs, only a limited number of studies are reported on human NMJs. Two studies on postmortem human muscle tissues revealed age-related fragmentation of endplates, which is common in aged rodents (Oda, 1984; Wokke et al, 1990). Contradicting this, another group studied the morphology of human NMJs using lower limb muscle tissues after amputation surgery, and reported that human NMJs throughout the adult lifespan (mean age, 67 years old; range, 34 to 92 years old) remain devoid of any of the age-related changes studied such as fragmentation of endplates, although denervation rate per se was not investigated (Jones et al, 2017). However, it might be better to interpret the data with some caution, because 1) the analyzed tissues were from patients with peripheral vascular disease or diabetes mellitus, and thus probably affected by their pathological conditions, and 2) diameter of myofibers or of motor axons in older age groups was not decreased while it is established that myofibers and motor axons degenerate as humans advance into old age (Azzabou et al, 2015; Cartee et al, 2016; Galbán et al, 2007). Furthermore, a recent study suggests that failure to reinnervate denervated myofibers leads to reduced muscle strength in patients with sarcopenia (Piasecki et al, 2018). Consistent with this, in rodents, it has been established that progressive NMJ denervation and reduction of muscle strength occur with ageing (Chai et al, 2011; Valdez et al, 2010). These observations suggest that age-related motor impairments are caused, at least in part, by NMJ denervation in mammals, although further investigations are required to fully understand age-related alterations of human NMJs.

As mentioned above, we previously demonstrated that AAV-D7 treatment enlarges NMJs, ameliorates the shortened life span and improves the impaired motor activity in mouse models of neuromuscular disorders with NMJ defects, such as DOK7 myasthenia, autosomal dominant Emery-Dreifuss muscular dystrophy, and ALS (Arimura et al, 2014; Miyoshi et al, 2017). In addition, other groups have shown that treatment with an agonist antibody to MuSK preserves NMJ innervation in a mouse model of ALS, although its effects on neuromuscular transmission and survival are controversial (Cantor et al, 2018; Sengupta-Ghosh et al, 2019). Furthermore, administration of a stabilized form (NT-1654) of the C-terminal 44 kDa fragment of motor neuron-derived agrin, a MuSK activator, enhances NMJ formation and improves motor behavior and survival of a mouse model of spinal muscular atrophy (Boido et al, 2018). These data together demonstrate that the NMJ is a promising therapeutic target in an array of neuromuscular diseases with NMJ defects. However, the effect of DOK7 gene therapy on age-related motor dysfunction was unknown. In the present study, we demonstrated that a single-dose systemic administration of AAV-D7 to 24 mo mice enhanced MuSK activation and NMJ innervation in the aged mice (FIGS. 1A, 1B, and 1E), and augmented motor function and muscle strength (FIGS. 3A and 3D). Therefore, DOK7 gene therapy exerts therapeutic effects on the muscle weakness and motor dysfunction of aged mice, although the mechanisms by which AAV-D7 treatment enhances NMJ innervation remain to be elucidated. We have previously shown that muscle-specific overexpression of Dok-7 induces the enlargement of not only the postsynaptic area but also the presynaptic motor nerve terminal (Inoue et al, 2009; Tezuka et al, 2014), suggesting that Dok-7 expression in muscle activates retrograde signaling from muscle to motor neurons. This retrograde signaling may contribute to NMJ innervation in aged mice by suppressing the degeneration of presynaptic motor nerve terminals and/or by accelerating the reinnervation of denervated myofiber.

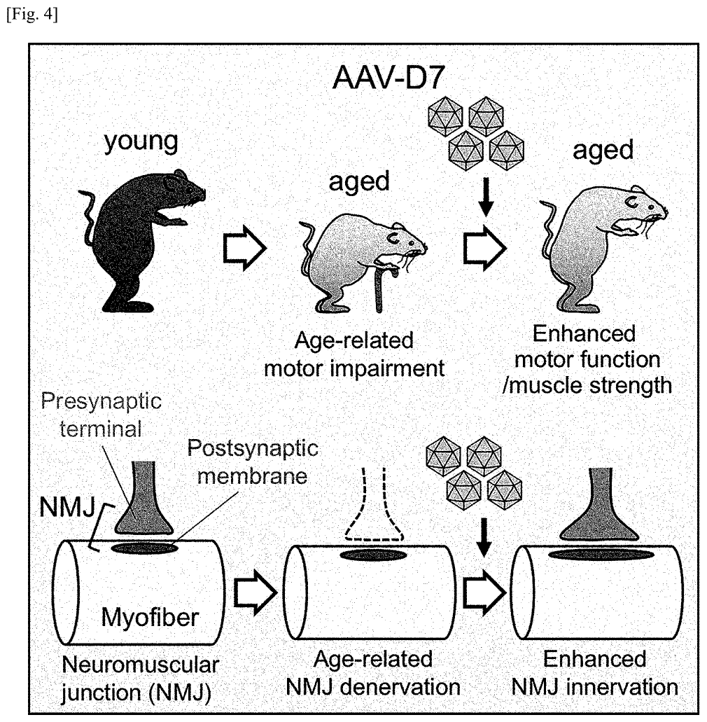

The formation and maintenance of NMJs require not only Dok-7, but also Lrp4, a receptor of agrin for MuSK activation and also an important retrograde signal to induce presynaptic specialization of motor nerve terminals at NMJs (Wu et al, 2012; Yumoto et al, 2012). A paper recently reported reduced levels of Lrp4 protein expression and MuSK activation in muscle of aged mice, suggesting impaired MuSK-mediated signaling due to loss of Lrp4 protein. Indeed, the authors demonstrated that transgenic expression of Lrp4 in skeletal muscle from the embryonic stage alleviates NMJ denervation, and improves neuromuscular transmission and muscle strength in aged mice (Zhao et al, 2018). Also, they found that Lrp4 interacts with sarcoglycan alpha (SG alpha) and demonstrated that intramuscular injection with an AAV serotype 9 vector expressing SG alpha fused with green fluorescent protein (GFP) (AAV9-SG alpha-GFP) at 22.5 months of age resulted in increased NMJ innervation, neuromuscular transmission, and muscle strength as compared with the AAV9-GFP-treated controls in 24 mo mice. However, they did not examine any enhancement of these values in comparison with each pre-dose value. Also, they examined neither MuSK activation nor motor function upon AAV9-SG alpha-GFP treatment or transgenic expression of Lrp4, unlike the present study on DOK7 gene therapy (FIGS. 1A and 3A). Interestingly, the transgenic expression of Lrp4 in muscle or administration of AAV9-SG alpha-GFP induced a significant increase in the CSA of the TA muscle in aged mice, in contrast to DOK7 gene therapy (FIGS. 3F and 3G), suggesting distinct mechanisms by which AAV-D7 administration enhances muscle strength. As mentioned, the decrease in muscle strength with ageing is known to be attributed not only to the decrease in muscle mass, but also to altered mechanical properties of myofiber; the force generated by myofibers normalized to their CSA (specific force) declines with ageing (Bruce et al, 1989; Gonzalez et al, 2000; Morse et al, 2005; Urbanchek et al, 2001). Thus, DOK7 gene therapy might increase the specific force by improving such mechanical properties of myofibers. In addition, because AAV-D7-treated aged mice showed increases in NMJ innervation and CMAP amplitude (FIGS. 1B, 1E, and 2), this treatment likely increases the number of functional NMJs, leading to enhanced muscle strength. Furthermore, because overexpression of Dok-7 in skeletal muscle enhances neuromuscular transmission at individual NMJs (Eguchi et al, 2020), AAV-D7 treatment may also strengthen neuromuscular transmission at individual NMJs in aged mice. Without wishing to be bound by any theory, FIG. 4 illustrates an exemplary mechanism for the treatment of age -related dysfunction with AAV-D7.

Limitations of the Study

However, DOK7 gene therapy has limitations in precise control of the duration and level of therapeutic intervention. Although this gene therapy benefited DOK7 myasthenia or aged mice for at least 12 or 4 months, respectively, with no apparent abnormality (Arimura et al, 2014; the present study), there remains a potential risk of sustained adverse side effects, such as genotoxicity, in aged humans (Wang et al, 2019). Thus, it would be important to develop compound-based treatment in parallel with gene therapy. However, because age-related motor dysfunction is a progressive, multifactorial disorder, NMJ-targeting therapy per se can serve only as a symptomatic treatment for the age-related dysfunction, but not as a cure. Given that several groups have developed treatments aimed at inducing muscle hypertrophy for age-related loss of skeletal muscle mass and subsequent motor dysfunction (Fujii et al, 2017; Vinel et al, 2018), DOK7 gene therapy or other NMJ-targeted therapies, might be more effective for age-related motor dysfunction when used in combination with other non-NMJ-targeting therapeutics, such as those aimed at inducing muscle hypertrophy.

Methods

All methods can be found in the accompanying Transparent Methods supplemental file.

Transparent Methods

Mice

Male C57BL6/N were purchased from Japan SLC and maintained in the Experimental Animal Facility of the National Center for Geriatrics and Gerontology (NCGG) until experimental use. Mice were housed on a 12/12-hour light/dark cycle in specific pathogen-free conditions with free access to water and standard mouse chows. All animal experiments were conducted in the Laboratory Animal Research Center of The Institute of Medical Science, The University of Tokyo, in the Experimental Animal Facility of NCGG, or in the Experimental Animal Facility of Kao Corporation's R&D Department. All animal studies were performed in accordance with the guidelines for animal care and use of each institute, and approved by the institutional animal care and use committees.

AAV Production and Injection

The cDNA encoding human Dok-7 cDNA tagged with c-myc epitope was cloned into pAAV-MCS (Agilent Technologies), which carries the cytomegalovirus promoter, to obtain pAAV-Dok-7-myc plasmid. For production of AAV-D7, HEK293EB cells were co-transfected with the AAV9 chimeric helper plasmid pRep2Cap9, the adenovirus helper plasmid pHelper (Agilent Technologies), and pAAV-MCS or pAAV-Dok-7-myc in a HYPERFlask vessel (Corning) using polyethylenimine, and cultured for 5 days (Lin et al, 2007; Matsushita et al, 2004). The AAV particles were purified by density-gradient ultracentrifugation (Tomono et al, 2016). The viral titers were determined by real-time quantitative PCR using AAVpro Titration Kit (Takara Bio). 4.8×10¹³ vg/kg body weight of AAV-D7 or AAV-ø were intravenously injected by a single dose via the tail vein.

Immunoprecipitation and Western Blotting

Tissue lysates were prepared from hindlimb muscle with alkaline lysis buffer [50 mM Tris·HCl (pH 9.5), 1% sodium deoxycholate, Complete protease inhibitor (Roche), PhosSTOP phosphatase inhibitor (Roche)]. For immunoprecipitation, lysates were incubated with antibodies to MuSK (N-19 and C-19) (Santa Cruz Biotechnology) or AChR betal (H-101) (Santa Cruz Biotechnology), followed by incubation with protein G-Sepharose (GE Healthcare). The immune complexes were washed five times and collected as immunoprecipitates. For Western blotting, immunoprecipitates or lysates were separated by SDS-PAGE on 6 or 9% gels and transferred to a PVDF membrane (Merck Millipore), which was then incubated with antibodies to phosphotyrosine (4G10) (Merck Millipore), MuSK (AF562) (R&D Systems), AChR betal (H-101) (Santa Cruz Biotechnology), Dok-7 (A-7) (Santa Cruz Biotechnology), or actin (I-19) (Santa Cruz Biotechnology), washed, and incubated with horseradish peroxidase-labeled anti-mouse (GE Healthcare) or anti-goat (Santa Cruz Biotechnology) IgG. The blots were visualized using a LAS4000 imager with ECL Prime Western Blotting Detection Reagent (GE Healthcare).

Immunohistochemistry of NMJs

Mice were anesthetized and perfused through the heart with PBS. Skeletal muscles were dissected out, embedded in Tissue-Tek OCT compound (Sakura Finetek) and processed for cryostat sectioning. 30-μm longitudinal cryosections were blocked in PBS containing 2% BSA and 0.1% Triton X-100. Sections were sequentially incubated with primary antibodies overnight at 4° C., washed with PBS, and incubated in a mixture of Alexa Fluor 488-conjugated secondary antibodies (Thermo Fischer Scientific) and CF 594-conjugated alpha-Bungarotoxin (Biotium) overnight at 4° C. After washing, the sections were mounted with Vectashield (Vector Laboratories). Confocal Z serial images were collected with an FV1000 Confocal Laser Scanning Microscope (Olympus) and collapsed into a single image. Images were captured with the same settings and exposure time in each experimental group for comparison. The sizes (areas) of presynaptic motor nerve terminals and postsynaptic AChR clusters were quantified using cellSens Digital Imaging Software (Olympus). For quantification, seven microscopic fields with the 20× objective were chosen at random on the tibialis anterior muscle from each mouse, and more than 100 synaptic sites were analyzed per mouse. These experiments were conducted in a blinded fashion.

Electromyography

Compound muscle action potentials (CMAPs) were studied using a PowerLab 26T data acquisition system (ADlnstruments). Mice were anesthetized using isoflurane inhalation, and the sciatic nerve was exposed at the left mid-thigh. Paired stimulating electrodes separated by 3 mm were kept in contact with the exposed sciatic nerve at 10 mm from the midline for supramaximal stimulation at 10 Hz. The recording electrodes were inserted in the middle of the left tibialis anterior muscle whereas the reference one was inserted 5-mm distally, both of which were connected via an MPA8I preamplifier (Multi Channel Systems) to an SC8×8BC signal collector (Multi Channel Systems). To isolate stimulus artifacts, a ground electrode was placed between the stimulus and recording electrodes. CMAPs were recorded, and peak—peak amplitudes were determined in LabChart software (ADlnstruments). These experiments were conducted in a blinded fashion.

Rotarod Test

Mice were placed on a rotating cylinder (MK-610A) (Muromachi Kikai), and the latency to fall was recorded. The device was set to accelerate from 4 to 40 rpm over a 5-min period. Before testing, each mouse was acclimated to the rotarod device for three trials per day on three consecutive days to familiarize the mice with the device and test protocols. The test was performed immediately before administration of the AAV and every 0.5 months thereafter until 2.5 months after the administration. The measurement was performed three times each day, and the average of the individual mice's measured values was calculated and estimated as the index of motor performance. These experiments were conducted in a blinded fashion.

Measurement of Maximal Plantarflexion Isometric Torque

Maximal plantarflexion isometric torque was measured with a slight modification of the method previously described (Itoh et al, 2017). Briefly, under anesthesia with isoflurane, electrical stimulation was applied to the posterior surface of the skin of the lower limbs. To attach surface stimulation electrodes (Bio Research Center) to the skin, viscous electrical conductive gel (CR) (Sekisui Plastics) was applied between the electrodes and the skin. The electrodes were fixed with adhesive tape to the surface of the myotendinous junction and a 5-mm proximal locus. Plantarflexor muscles were percutaneously stimulated via surface stimulation electrodes, and maximal plantarflexion was evoked using a supramaximal twitch current (100-Hz frequency, 1.0-msec duration, and 10.0-mA current). Isometric plantarflexion torque (T) was calculated from the pressure applied to a footplate (F) and the distance from the axis of the ankle joint to the sensor (r) as follows: T=Fr. These experiments were conducted in a blinded fashion.

### REFERENCES

The following references are herein incorporated by reference in their entirety with the exception that, should the scope and meaning of a term conflict with a definition explicitly set forth herein, the definition explicitly set forth herein controls:

Andonian, M. H., and Fahim, M. A. (1987). Effects of endurance exercise on the morphology of mouse neuromuscular junctions during ageing. J. Neurocytol. 16, 589-599.

Arimura, S., Okada, T., Tezuka, T., Chiyo, T., Kasahara, Y., Yoshimura, T., Motomura, M., Yoshida, N., Beeson, D., Takeda, S., et al. (2014). DOK7 gene therapy benefits mouse models of diseases characterized by defects in the neuromuscular junction. Science 345, 1505-1508.

Azzabou N., Hogrel J-Y., and Carlier P. G. (2015). NMR based biomarkers to study age-related changes in the human quadriceps. Exp. Gerontol. 70, 54-60.

Beeson, D., Higuchi, O., Palace, J., Cossins, J., Spearman, H., Maxwell, S., Newsom-Davis, J.,

Burke, G., Fawcett, P., Motomura, M., et al. (2006). Dok-7 mutations underlie a neuromuscular junction synaptopathy. Science. 313, 1975-1978.

Boido, M., de Amicis, E., Valsecchi, V., Trevisan, M., Ala, U., Ruegg, M. A., Hettwer, S., and Vercelli, A. (2018). Increasing agrin function antagonizes muscle atrophy and motor impairment in spinal muscular atrophy. Front. Cell. Neurosci. 12, 1-14.

Bruce, S. A., Newton, D., and Woledge, R. C. (1989). Effect of Age on Voluntary Force and Cross-Sectional Area of Human Adductor Pollicis Muscle. Q. J. Exp. Physiol. 74, 359-362.

Burden, S.J. (2002). Building the vertebrate neuromuscular synapse. J. Neurobiol. 53, 501-511.

Bütikofer, L., Zurlinden, A., Bolliger, M. F., Kunz, B., and Sonderegger, P. (2011). Destabilization of the neuromuscular junction by proteolytic cleavage of agrin results in precocious sarcopenia. FASEB J. 25, 4378-4393.

Campbell, M. J., McComas, A. J., and Petito, F. (1973). Physiological changes in ageing muscles. J. Neurol. Neurosurg. Psychiatry 36, 174-182.

Cantor, S., Zhang, W., Delestrée, N., Remédio, L., Mentis, G. Z., and Burden, S. J. (2018). Preserving neuromuscular synapses in ALS by stimulating MuSK with a therapeutic agonist antibody. Elife 7, 1-19.

Cartee, G. D., Hepple, R. T., Bamman, M. M., and Zierath, J. R. (2016). Exercise promotes healthy aging of skeletal muscle. Cell Metab. 23, 1034-1047.

Chai, R. J., Vukovic, J., Dunlop, S., Grounds, M. D., and Shavlakadze, T. (2011). Striking denervation of neuromuscular junctions without lumbar motoneuron loss in geriatric mouse muscle. PLoS One 6, e28090.

Cheng, A., Morsch, M., Murata, Y., Ghazanfari, N., Reddel, S. W., and Phillips, W.D. (2013). Sequence of age-associated changes to the mouse neuromuscular junction and the protective effects of voluntary exercise. PLoS One 8, e67970.

Didier, N., Hourdé, C., Amthor, H., Marazzi, G., and Sassoon, D. (2012). Loss of a single allele for Ku80 leads to progenitor dysfunction and accelerated aging in skeletal muscle. EMBO Mol. Med. 4, 910-923.

Eguchi, T., Tezuka, T., Fukudome, T., Watanabe, Y., Sagara, H., and Yamanashi, Y. (2020). Overexpression of Dok-7 in skeletal muscle enhances neuromuscular transmission with structural alterations of neuromuscular junctions: Implications in robustness of neuromuscular transmission. Biochem. Biophys. Res. Commun. 523, 214-219.

Fuhrer, C., Sugiyama, J. E., Taylor, R. G., and Hall, Z. W. (1997). Association of muscle-specific kinase MuSK with the acetylcholine receptor in mammalian muscle. EMBO J. 16, 4951-4960.

Fujii, C., Miyashita, K., Mitsuishi, M., Sato, M., Fujii, K., Inoue, H., Hagiwara, A., Endo, S., Uto, A., Ryuzaki, M., et al. (2017). Treatment of sarcopenia and glucose intolerance through mitochondrial activation by 5-aminolevulinic acid. Sci. Rep. 7, 1-12.

Galbán, C. J., Maderwald, S., Stock, F., and Ladd, M. E. (2007). Age-related changes in skeletal muscle as detected by diffusion tensor magnetic resonance imaging. J. Gerontol. Ser. A. Biol. Sci. Med. Sci. 62, 453-458.

González-Freire, M., de Cabo, R., Studenski, S. A., and Ferrucci, L. (2014). The neuromuscular junction: Aging at the crossroad between nerves and muscle. Front. Aging Neurosci. 6, 208.

González, E., Messi, M. L., and Delbono, O. (2000). The specific force of single intact extensor digitorum longus and soleus mouse muscle fibers declines with aging. J. Membr. Biol. 178, 175-183.

Graber, T. G., Ferguson-Stegall, L., Kim, J. H., and Thompson, L. V (2013). C57BL/6 neuromuscular health span scoring system. J. Gerontol. A. Biol. Sci. Med. Sci. 68, 1326-1336.

Günther, R., Suhr, M., Koch, J. C., Bahr, M., Lingor, P., and Tonges, L. (2012). Clinical testing and spinal cord removal in a mouse model for amyotrophic lateral sclerosis (ALS). J. Vis. Exp. (61), e3936.

Guth, L., Albers, R. W., and Brown, W. C. (1964). Quantitative changes in cholinesterase activity of denervated muscle fibers and sole plates. Exp. Neurol. 10, 236-250.

Hirofuji, C., Ishihara, A., Roy, R. R., Itoh, K., Itoh, M., Edgerton, V. R., and Katsuta, S. (2000). SDH activity and cell size of tibialis anterior motoneurons and muscle fibers in SAMP6. Neuroreport 11, 823-828.

Hunter, S. K., Pereira, X. H. M., and Keenan, K. G. (2016). The aging neuromuscular system and motor performance. J. Appl. Physiol. 121, 982-995.

Inoue, A., Setoguchi, K., Matsubara, Y., Okada, K., Sato, N., Iwakura, Y., Higuchi, O., and Yamanashi, Y. (2009). Dok-7 activates the muscle receptor kinase MuSK and shapes synapse formation. Sci. Signal. 2, ra7.

Itoh, Y., Murakami, T., Mori, T., Agata, N., Kimura, N., Inoue-Miyazu, M., Hayakawa, K., Hirano, T., Sokabe, M., and Kawakami, K. (2017). Training at non-damaging intensities facilitates recovery from muscle atrophy. Muscle Nerve 55,243-253.

Jones, R. A., Harrison, C., Eaton, S. L., Llavero Hurtado, M., Graham, L. C., Alkhammash, L., Oladiran, O. A., Gale, A., Lamont, D. J., Simpson, H., et al. (2017). Cellular and Molecular Anatomy of the Human Neuromuscular Junction. Cell Rep. 21, 2348-2356.

Kim, N., Stiegler, A. L., Cameron, T. O., Hallock, P. T., Gomez, A. M., Huang, J. H., Hubbard, S. R., Dustin, M. L., and Burden, S. J. (2008). Lrp4 is a receptor for Agrin and forms a complex with MuSK. Cell 135, 334-342.

Kurokawa, K., Mimori, Y., Tanaka, E., Kohriyama, T., and Nakamura, S. (1999). Age-related change in peripheral nerve conduction: Compound muscle action potential duration and dispersion. Gerontology 45, 168-173.

Lexell, J., and Downham, D. Y. (1991). The occurrence of fibre-type grouping in healthy human muscle: a quantitative study of cross-sections of whole vastus lateralis from men between 15 and 83 years. Acta Neuropathol. 81,377-381.

Zhi, Y., Mays, L., and Wilson, J. M. (2007). Vaccines Based on Novel Adeno-Associated Virus Vectors Elicit Aberrant CD8⁺T-Cell Responses in Mice. J. Virol. 81, 11840-11849.

Liu, W., Klose, A., Forman, S., Paris, N. D., Wei-LaPierre, L., Cortes-Lopez, M., Tan, A., Flaherty, M., Miura, P., Dirksen, R. T., et al. (2017). Loss of adult skeletal muscle stem cells drives age-related neuromuscular junction degeneration. Elife 6, e26464.

Matsushita, T., Okada, T., Inaba, T., Mizukami, H., Ozawa, K., and Colosi, P. (2004). The adenovirus E1A and E1B19K genes provide a helper function for transfection-based adeno-associated virus vector production. J. Gen. Virol. 85, 2209-2214.

Méjat, A., Decostre, V., Li, J., Renou, L., Kesari, A., Hantaë, D., Stewart, C. L., Xiao, X., Hoffman, E., Bonne, G., et al. (2009). Lamin A/C—mediated neuromuscular junction defects in Emery-Dreifuss muscular dystrophy. J. Cell Biol. 184, 31-44.

Miyoshi, S., Tezuka, T., Arimura, S., Tomono, T., Okada, T., and Yamanashi, Y. (2017). DOK7 gene therapy enhances motor activity and life span in ALS model mice. EMBO Mol. Med. 9, 880-889.

Morse, C. I., Thom, J. M., Reeves, N. D., Birch, K. M., and Narici, M. V. (2005). In vivo physiological cross-sectional area and specific force are reduced in the gastrocnemius of elderly men. J. Appl. Physiol. 99, 1050-1055.

Oda, K. (1984). Age changes of motor innervation and acetylcholine receptor distribution on human skeletal muscle fibres. J. Neurol. Sci. 66, 327-338.

Okada, K., Inoue, A., Okada, M., Murata, Y., Kakuta, S., Jigami, T., Kubo, S., Shiraishi, H., Eguchi, K., Motomura, M., et al. (2006). The muscle protein Dok-7 is essential for neuromuscular synaptogenesis. Science. 312, 1802-1805.

Pannérec, A., Springer, M., Migliavacca, E., Ireland, A., Piasecki, M., Karaz, S., Jacot, G., Métairon, S., Danenberg, E., Raymond, F., et al. (2016). A robust neuromuscular system protects rat and human skeletal muscle from sarcopenia. Aging (Albany. N.Y.). 8, 712-729.

Piasecki, M., Ireland, A., Piasecki, J., Stashuk, D. W., Swiecicka, A., Rutter, M. K., Jones, D. A., and McPhee, J. S. (2018). Failure to expand the motor unit size to compensate for declining motor unit numbers distinguishes sarcopenic from non-sarcopenic older men. J. Physiol. 596, 1627-1637.

Punga, A. R., and Ruegg, M. A. (2012). Signaling and aging at the neuromuscular synapse: Lessons learnt from neuromuscular diseases. Curr. Opin. Pharmacol. 12, 340-346.

Sadakata, T., Kakegawa, W., Mizoguchi, A., Washida, M., Katoh-Semba, R., Shutoh, F., Okamoto, T., Nakashima, H., Kimura, K., Tanaka, M., et al. (2007). Impaired cerebellar development and function in mice lacking CAPS2, a protein involved in neurotrophin release. J. Neurosci. 27, 2472-2482.

Sengupta-Ghosh, A., Dominguez, S. L., Xie, L., Barck, K. H., Jiang, Z., Earr, T., Imperio, J., Phu, L., Budayeva, H. G., Kirkpatrick, D. S., et al. (2019). Muscle specific kinase (MuSK) activation preserves neuromuscular junctions in the diaphragm but is not sufficient to provide a functional benefit in the SOD1G93A mouse model of ALS. Neurobiol. Dis. 124, 340-352.

Spendiff, S., Vuda, M., Gouspillou, G., Aare, S., Perez, A., Morais, J. A., Jagoe, R. T., Filion, M. E., Glicksman, R., Kapchinsky, S., et al. (2016). Denervation drives mitochondrial dysfunction in skeletal muscle of octogenarians. J. Physiol. 594, 7361-7379.

Taetzsch, T., and Valdez, G. (2018). NMJ maintenance and repair in aging. Curr. Opin. Pharmacol. 4, 57-64.

Tezuka, T., Inoue, A., Hoshi, T., Weatherbee, S. D., Burgess, R. W., Ueta, R., and Yamanashi, Y. (2014). The MuSK activator agrin has a separate role essential for postnatal maintenance of neuromuscular synapses. Proc. Natl. Acad. Sci. USA 111, 16556-16561.

Tintignac, L. A., Brenner, H. R., and Rüegg, M. A. (2015). Mechanisms Regulating Neuromuscular Junction Development and Function and Causes of Muscle Wasting. Physiol. Rev. 95, 809-852.

Tomono, T., Hirai, Y., Okada, H., Adachi, K., Ishii, A., Shimada, T., Onodera, M., Tamaoka, A., and Okada, T. (2016). Ultracentrifugation-free chromatography-mediated large-scale purification of recombinant adeno-associated virus serotype 1 (rAAV1). Mol. Ther. Methods Clin. Dev. 3,15058.

Turturro, A., Witt, W. W., Lewis, S., Hass, B. S., Lipman, R. D., and Hart, R. W. (1999). Growth curves and survival characteristics of the animals used in the biomarkers of aging program. J. Gerontol. A. Biol. Sci. Med. Sci. 54, 492-501.

Urbanchek, M. G., Picken, E. B., Kalliainen, L. K., and Kuzon, W. M. (2001). Specific force deficit in skeletal muscles of old rats is partially explained by the existence of denervated muscle fibers. J. Gerontol. A. Biol. Sci. Med. Sci. 56, B191—B197.

Valdez, G., Tapia, J. C., Kang, H., Clemenson, G. D., Gage, F. H., Lichtman, J. W., and Sanes, J. R. (2010). Attenuation of age-related changes in mouse neuromuscular synapses by caloric restriction and exercise. Proc. Natl. Acad. Sci. USA 107, 14863-14868.

Valdez, G., Tapia, J. C., Lichtman, J. W., Fox, M. A., and Sanes, J. R. (2012). Shared resistance to aging and ALS in neuromuscular junctions of specific muscles. PLoS One 7, e34640.

Vinel, C., Lukjanenko, L., Batut, A., Deleruyelle, S., Pradère, J. P., Le Gonidec, S., Dortignac, A., Geoffre, N., Pereira, O., Karaz, S., et al. (2018). The exerkine apelin reverses age-associated sarcopenia. Nat. Med. 24, 1360-1371.

Wang, D., Tai, P. W. L., and Gao, G. (2019). Adeno-associated virus vector as a platform for gene therapy delivery. Nat. Rev. Drug Discov. 18, 358-378.

Willadt, S., Nash, M., and Slater, C. (2018). Age-related changes in the structure and function of mammalian neuromuscular junctions. Ann. N. Y. Acad. Sci. 1412, 41-53.

Wokke, J. H., Jennekens, F. G., van den Oord, C. J., Veldman, H., Smit, L. M., and Leppink, G. J. (1990). Morphological changes in the human end plate with age. J. Neurol. Sci. 95, 291-310.

Wu, H., Lu, Y., Shen, C., Patel, N., Gan, L., Xiong, W. C., and Mei, L. (2012). Distinct roles of muscle and motoneuron LRP4 in neuromuscular junction formation. Neuron 75, 94-107.

Wu P. B., Gussner C. G., and Date E. S. (1996). Correlation of EMG, CMAP and SNAP amplitude decrease in mononeuropathies with axonal loss. Electromyogr. Clin. Neurophysiol. 36, 405-409.

Yumoto, N., Kim, N., and Burden, S. J. (2012). Lrp4 is a retrograde signal for presynaptic differentiation at neuromuscular synapses. Nature 489, 438-442.

Zhang, B., Luo, S., Wang, Q., Suzuki, T., Xiong, W. C., and Mei, L. (2008). LRP4 serves as a coreceptor of agrin. Neuron 60, 285-297.

Zhao, K., Shen, C., Li, L., Wu, H., Xing, G., Dong, Z., Jing, H., Chen, W., Zhang, H., Tan, Z., et al. (2018). Sarcoglycan Alpha Mitigates Neuromuscular Junction Decline in Aged Mice by Stabilizing LRP4. J. Neurosci. 38, 8860-8873.

All scientific and technical terms used in this application have meanings commonly used in the art unless otherwise specified.

As used herein, a given percentage of “sequence identity” refers to the percentage of nucleotides or amino acid residues that are the same between sequences, when compared and optimally aligned for maximum correspondence over a given comparison window, as measured by visual inspection or by a sequence comparison algorithm in the art, such as the BLAST algorithm, which is described in Altschul et al., J. Mol. Biol. 215:403-410 (1990). Software for performing BLAST (e. g., BLASTP and BLASTN) analyses is publicly available through the National Center for Biotechnology Information (ncbi.nlm.nih.gov). The comparison window can exist over a given portion, e. g., a functional domain, or an arbitrarily selection a given number of contiguous nucleotides or amino acid residues of one or both sequences. Alternatively, the comparison window can exist over the full length of the sequences being compared. For purposes herein, where a given comparison window (e. g., over 80% of the given sequence) is not provided, the recited sequence identity is over 100% of the given sequence. Additionally, for the percentages of sequence identity of the proteins provided herein, the percentages are determined using BLASTP 2.8.0+, scoring matrix BLOSUM62, and the default parameters available at blast.ncbi.nlm.nih.gov/Blast. cgi. See also Altschul, et al. (1997), Nucleic Acids Res. 25:3389-3402; and Altschul, et al. (2005) FEBS J. 272:5101-5109.

Optimal alignment of sequences for comparison can be conducted, e. g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection.

As used herein, the terms “protein”, “polypeptide” and “peptide” are used interchangeably to refer to two or more amino acids linked together. Groups or strings of amino acid abbreviations are used to represent peptides. Except when specifically indicated, peptides are indicated with the N-terminus on the left and the sequence is written from the N-terminus to the C-terminus.

As used herein, “antibody” refers to naturally occurring and synthetic immunoglobulin molecules and immunologically active portions thereof (i. e., molecules that contain an antigen binding site that specifically bind the molecule to which antibody is directed against). As such, the term antibody encompasses not only whole antibody molecules, but also antibody multimers and antibody fragments as well as variants (including derivatives) of antibodies, antibody multimers and antibody fragments. Examples of molecules which are described by the term “antibody” herein include: single chain Fvs (scFvs), Fab fragments, Fab′ fragments, F(ab′)2, disulfide linked Fvs (sdFvs), Fvs, and fragments comprising or alternatively consisting of, either a VL or a VH domain.

As used herein, a compound (e. g., receptor or antibody) “specifically binds” a given target (e. g., ligand or epitope) if it reacts or associates more frequently, more rapidly, with greater duration, and/or with greater binding affinity with the given target than it does with a given alternative, and/or indiscriminate binding that gives rise to non-specific binding and/or background binding. As used herein, “non-specific binding” and “background binding” refer to an interaction that is not dependent on the presence of a specific structure (e. g., a given epitope).

As used herein, the terms “subject”, “patient”, and “individual” are used interchangeably to refer to humans and non-human animals. The term “non-human animal” includes all vertebrates, e. g., mammals and non-mammals, such as non-human primates, horses, sheep, dogs, cows, pigs, chickens, and other veterinary subjects and test animals. In some embodiments of the present invention, the subject is a mammal. In some embodiments of the present invention, the subject is a human.

As used herein, the term “diagnosing” refers to the physical and active step of informing, i. e., communicating verbally or by writing (on, e. g., paper or electronic media), another party, e. g., a patient, of the diagnosis. Similarly, “providing a prognosis” refers to the physical and active step of informing, i. e., communicating verbally or by writing (on, e. g., paper or electronic media), another party, e. g., a patient, of the prognosis.

The use of the singular can include the plural unless specifically stated otherwise. As used in the specification and the appended claims, the singular forms “a”, “an”, and “the” can include plural referents unless the context clearly dictates otherwise.

As used herein, “and/or” means “and” or “or”. For example, “A and/or B” means “A, B, or both A and B” and “A, B, C, and/or D” means “A, B, C, D, or a combination thereof” and said “A, B, C, D, or a combination thereof” means any subset of A, B, C, and D, for example, a single member subset (e. g., A or B or C or D), a two-member subset (e. g., A and B; A and C; etc. ), or a three-member subset (e. g., A, B, and C; or A, B, and D; etc. ), or all four members (e. g., A, B, C, and D).

As used herein, the phrase “one or more of”, e. g., “one or more of A, B, and/or C” means “one or more of A”, “one or more of B”, “one or more of C”, “one or more of A and one or more of B”, “one or more of B and one or more of C”, “one or more of A and one or more of C” and “one or more of A, one or more of B, and one or more of C”.

The phrase “comprises or consists of A” is used as a tool to avoid excess page and translation fees and means that in some embodiments the given thing at issue: comprises A or consists of A. For example, the sentence “In some embodiments, the composition comprises or consists of A” is to be interpreted as if written as the following two separate sentences: “In some embodiments, the composition comprises A. In some embodiments, the composition consists of A. ”

Similarly, a sentence reciting a string of alternates is to be interpreted as if a string of sentences were provided such that each given alternate was provided in a sentence by itself. For example, the sentence “In some embodiments, the composition comprises A, B, or C” is to be interpreted as if written as the following three separate sentences: “In some embodiments, the composition comprises A. In some embodiments, the composition comprises B. In some embodiments, the composition comprises C. ” As another example, the sentence “In some embodiments, the composition comprises at least A, B, or C” is to be interpreted as if written as the following three separate sentences: “In some embodiments, the composition comprises at least A. In some embodiments, the composition comprises at least B. In some embodiments, the composition comprises at least C.”

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1. A method for the treatment of age-related dysfunction in a subject in need thereof, the method comprising administering to the subject a therapeutically effective amount of an NMJ (neuromuscular junction) targeting agent.

2. The method according to claim 1, wherein said subject is an elderly subject without a disease associated with defective NMJs.

3. The method according to claim 2, wherein said disease is one or more neuromuscular disorders selected from the group consisting of congenital myasthenic syndromes (CMS), myasthenia gravis (MG), muscular dystrophy (MD), amyotrophic lateral sclerosis (ALS), spinal muscular atrophy (SMA) and Charcot-Marie-Tooth disease type 2D.

4. The method according to claim 1, wherein said age-related dysfunction is age-related motor impairment which is associated with one or more symptoms selected from muscle weakness and motor dysfunction.

5. The method according to claim 1, wherein said age-related dysfunction is from age-related muscle atrophy or sarcopenia.

6. The method according to claim 1, wherein said agent is a polynucleotide which encodes Dok-7.

7. The method according to claim 1, wherein said treatment is effective to improve the motor function or muscle strength.

8. The method according to claim 7, wherein said function or muscle strength is significantly improved in comparison with a younger elderly subject without a disease associated with defective NMJs.

9. The method according to claim 1, wherein said treatment is effective to reduce age-related NMJ denervation.

10. The method according to claim 9, wherein said NMJ denervation is significantly reduced in comparison with a younger elderly subject without a disease associated with defective NMJs.

11. The method according to claim 1, wherein said NMJ targeting agent is used in combination with non-NMJ-targeting therapeutics.

12. A pharmaceutical composition for the treatment of age-related dysfunction in a subject in need thereof, the composition comprising a therapeutically effective amount of an NMJ targeting agent.

13-16. (canceled)

17. The pharmaceutical composition according to claim 12, wherein said agent is a polynucleotide which encodes Dok-7.

18-21. (canceled)

22. The pharmaceutical composition according to claim 12, wherein said NMJ targeting agent is used in combination with non-NMJ-targeting therapeutics.

23-27. (canceled)

28. An expression vector comprising a polynucleotide which encodes Dok-7.

29-33. (canceled)
